(19) United States
(12) Patent Application Publication
(10) Pub. No.: US 2023/0293396 A1
(43) Pub. Date: Sep. 21, 2023

(54) Method Of Improving Quality Of Foam Produced From Concentrated Foaming Hand Wash Compositions Comprising Benzoic Acid Preservative

(71) Applicant: Henkel AG & Co. KGaA, Dusseldorf (DE)

(72) Inventors: Valerie M. Gendelsman, Stamford, CT (US); Jeffrey S. Bankers, Stamford, CT (US); Jennifer S. Haghpanah, New York, NY (US); Phi H. Doan, Stamford, CT (US); Derek Doowon Kim, Fort Lee, NJ (US)

(21) Appl. No.: 17/696,483

(22) Filed: Mar. 16, 2022

(51) Int. Cl. A61K 8/04; A61Q 19/10; A61K 8/368; A61K 8/86; A61K 8/49

(52) U.S. Cl. CPC A61K 8/046; A61Q 19/10; A61K 8/368; A61K 8/86; A61K 8/4993

(57) ABSTRACT

A method of improving foam quality of foams prepared from a concentrated cleaning composition containing fragrance and benzoic acid includes mixing the fragrance with a hydrophilic solubilizer to form a premix and adding the premix to other ingredients of the concentrated composition. A ratio of fragrance to stabilizer in the concentrated composition is about 1:1 to about 3:1. The concentrated compositions are foamed by diluting with water to form a ready-to-use composition and mixing the ready-to-use composition with air in a mechanical foaming head.

### FIELD OF THE INVENTION

The invention relates to a concentrated foaming cleaning composition, a ready-to-use foaming cleaning composition, and a process of improving foam quality of a concentrated foaming cleaning composition. More particularly, the present invention relates to foaming hand wash compositions containing fragrance and benzoic acid preservative that are prepared by diluting a concentrated composition and mixing it with air, and a method for preparing the concentrated composition to improve foam characteristics.

### BACKGROUND OF THE INVENTION

Standard foaming hand washes are low-solids containing hand soap dispensed through a foamer pump. These formulas are commonly sold in retail stores and are single use purchases.

The foamer pump contains a chamber/housing and a mesh screen. The liquid formulation is drawn up into the chamber and extruded through the mesh, which aerates the surfactant-containing formulation before it is dispensed from the actuator. The mesh screen is typically between size 60- and 250-mesh (58-250 microns, respectively) and composed of nylon. Foam is created when gas is trapped within a liquid to create bubbles. When the liquid formulation is extruded through the mesh, air becomes entrapped within the liquid. Surfactants stabilize the foam through adsorption to the gas phase. This surface process creates the foam that a consumer expects from a foaming hand wash product.

Consumers expect a creamy and stable foam upon dispensation. The quality of a foam is subjective, but can be compared using foam stability, foam density, and bubble size distribution. A high-quality foam should be stable and dense with a narrow bubble size distribution. A high-quality foam should have a bubble count of at least 40 mm-2 and a mean bubble area of at most 25000 μm.

However, delivery of liquid cleansing compositions through foam producing dispensers has presented many challenges. Additives can interfere with the ability of the composition to foam. Furthermore, certain types of foam-producing dispensers that use porous filters or meshed screens to produce foam may not work well (or at all) with even moderately viscous compositions.

Consumers increasingly desire wash compositions that are stabilized with benzoic acid, as opposed to parabens, DMDM hydantoin, methylchloroisothiazolinone (MCIT), methylisothiazolinone (MIT), etc. However, use of benzoic acid salts, such as sodium benzoate, are more difficult to formulate in a wash composition and require a narrower pH range compared to other alternatives. Above pH 5.5, benzoic acid will remain bound in the salt form and have little to no preservative effect. On the other side, from a regulatory perspective, it is desired keep pH above 3. Adding fragrance can further complicate the formulation of such compositions.

Concentrated liquid cleansing formulations are a relatively new product category. These concentrated products are meant to improve the sustainability profile of liquid cleansers by reducing the amount of water shipped by the manufacturer and by reducing the amount of single-use plastic used by the consumer. Consumers are interested in the concentrated products due to increased concerns over their environmental impact.

Concentrated formulations contain less water before dilution when compared to a standard foaming wash formula, allowing for more chemical interactions between the raw materials. There is a desire to make concentrated cleaning foams that are preserved with benzoic acid or a salt thereof and that contain fragrance. However, it was discovered that attempting to concentrate one of Applicant's foaming hand wash formulas containing fragrance and sodium benzoate led to a chemical interaction in the concentrated form between the fragrance and sodium benzoate. This chemical interaction caused the mesh screen of a foamer pump to clog, undesirably reducing the foam density and broadening the bubble size distribution. The ready-to-use liquid passing through the pump is important for this product type because the mesh screen within the pump creates the desirable, evenly dispersed aeration.

It was an object of the invention to prevent some of the undesirable chemical interactions between sodium benzoate/benzoic acid and fragrance in a concentrated hand wash composition from occurring, which allows for the ready-to-use wash liquid to pass through the foaming hand wash pump and produce desirable foam characteristics. It was an object of the invention to provide consumers with sustainable foam wash composition concentrates containing sodium benzoate and fragrance with comparable performance to existing non-concentrated formulations. Namely, it was an object of the invention to provide concentrated handwash compositions containing sodium benzoate and fragrance that had a bubble count per mm² of above 25 and a mean bubble area below 40,000 μm².

### SUMMARY OF THE INVENTION

The foregoing objectives are achieved by a concentrated foaming cleaning composition comprising benzoic acid or a salt thereof, fragrance, cationic compatible surfactant, and a hydrophilic solubilizer. The fragrance to solubilizer ratio is preferably about 1:1 to about 3:1, more preferably between 1.5:1 to about 2.7:1.

A method of manufacturing and/or stabilizing a concentrated foaming handwash composition containing fragrance and benzoic acid preservative comprises mixing fragrance with a hydrophilic solubilizer to form a premix, and then adding the premix to a concentrated cleaning composition comprising benzoic acid or a salt thereof, a cationic compatible surfactant, and about 45 to about 60 percent by weight water. Preferably, the fragrance to solubilizer ratio is about 1:1 to about 3:1, more preferably between 1.5:1 to about 2.7:1.

A method of foaming a concentrated cleaning composition comprising benzoic acid or a salt thereof, fragrance, at least one cationic compatible surfactant, and a hydrophilic solubilizer is provided. The method includes the steps of diluting the concentrated foaming cleaning composition to form a ready-to-use composition and mixing the ready-to-use composition with air in a mechanical foaming head to generate foam.

Advantageously and unexpectedly, the ready-to-use cleaning composition produces a foam having a greater bubble count per mm² and a smaller mean bubble area than a foam prepared from a concentrated cleaning composition having no hydrophilic solubilizer or a concentrated composition having no fragrance.

### BRIEF DESCRIPTION OF THE DRAWINGS

FIG. 1A shows an exemplary foaming pump dispenser that can be used with the concentrated cleaning compositions described herein to generate a foam.

FIG. 1B shows the pump head of the foaming dispenser of Example 1A.

FIG. 2A shows foam structure of a commercial antibacterial, ready-to-use hand foaming hand wash formula containing fragrance that has a high quality foam.

FIG. 2B is a histogram of bubble count and size distribution of the comparative hand wash shown in FIG. 2A.

FIG. 3 shows images of foams produced according to Example 2. The y axis shows weight percent of fragrance in the concentrated composition and the x axis shows the dilution ratio of the concentrated composition to water.

FIG. 4 shows images of foams produced according to Example 2 at two different sodium benzoate levels.

FIG. 5 shows the particle size distribution of the foams of Example 3 obtained using a Dynamic Light Scattering (DLS) method.

FIG. 6 shows fluorescence intensity of the foams of Example 3.

FIG. 7 shows images of foams produced according to Example 3.

FIG. 8 shows images of the foams of Example 4 along with a bubble size histogram obtained using a Kruss Dynamic Foam Analyzer DFA 100.

### DETAILED DESCRIPTION OF THE INVENTION

The following description provides specific details, such as materials and amounts, to provide a thorough understanding of the present invention. The skilled artisan, however, will appreciate that the present invention can be practiced without employing these specific details. Indeed, the present invention can be practiced in conjunction with processing, manufacturing or fabricating techniques conventionally used in the detergent composition industry.

Concentrated compositions described herein comprise benzoic acid or a salt thereof, fragrance, at least one cationic compatible surfactant, which may encompass cationic, nonionic, zwitterionic, or amphoteric surfactants; a solubilizer/foam structure enhancing agent; and water. The concentrated compositions are prepared by mixing the fragrance with a hydrophilic solubilizer to form a fragrance premix and adding the fragrance premix to a composition comprising benzoic acid or a salt thereof, at least one cationic compatible surfactant. A method of foaming such concentrated compositions comprises diluting the concentrated composition to form a ready-to-use composition that can be mixed with air to create a foam. The concentrated compositions contain less than 80% by weight, more preferably less than 70% by weight, most preferably less than 65% by weight water.

As used herein, HLB refers to a value that is used to index and describe a surfactant according to its relative hydrophobicity/hydrophilicity, relative to other surfactants. A surfactant's HLB value is an indication of the molecular balance of the hydrophobic and lipophilic portions of the surfactant, which is an amphipathic molecule.

Absent explicit statement to the contrary, reference to wt. %, or wt %, or percent by weight, in the specification refers to the weight percentage of an ingredient as compared to the total weight of the cleaning composition. The wt. % of the total water in the liquid composition is calculated based on all the water including those added as a part of individual ingredients. When an ingredient added to make the liquid composition is not 100% pure and used as a mixture, e.g., in a form of a solution, the wt. % of that material added refers to the weight percentage of the mixture. Thus, a component which is 5 wt. % of the formulation, may be added as 5 wt. % of a pure component or 10 wt. % of solution that is 50% component and 50% water. Either result produces the recited 5 wt. % amount of the component in the resulting formulation. All percentages presented in this specification and the associated claims are weight percentages unless explicitly identified otherwise. If not indicated otherwise, all percentages refer to active matter and are by weight relative to the total weight of the composition.

Mole fractions and volume fractions are not used unless explicitly identified.

“At least one”, as used herein, relates to one or more, i.e., 1, 2, 3, 4, 5, 6, 7, 8, 9, or more. If used in combination with a compound, the term does not relate to the absolute number of molecules but rather to the number of different types of said compound. “At least one surfactant” thus means that at least one type but that also 2 or more different surfactant types can be present.

“About”, as used herein in relation to a numerical value, relates to said value ±10%, preferably ±5%. “About 2.0” thus refers to a range from 1.8 to 2.2, preferably from 1.9 to 2.1.

The cleaning compositions of the present invention can be used to cleanse the skin, especially the hands, arms, and face of the user. The phrase “cleaning composition” refers to a composition that provides for the removal of a substance from a surface to be cleaned. Exemplary substances that can be removed by the cleaning composition include soil, dirt, oil, grease, bacteria, microbes, viruses, etc. The cleaning composition can also be referred to as a detergent composition.

A concentrated cleaning composition can be referred to as a concentrate, and can be diluted to provide a ready-to-use cleaning composition and/or detergent use composition. The concentrate can be diluted in a single dilution or in stages to provide the ready-to-use cleaning composition and/or the detergent use composition. Providing the cleaning composition as a concentrate for subsequent dilution can be advantageous when it is desirable to package, ship, and sell the concentrate instead of the ready-to-use detergent composition and/or the detergent use composition. This eliminates unnecessary resell of bulky dispensing foaming devices. A foaming dispenser can be reused and the ready-to-use detergent composition can be made available as a use composition at a user's desire. This reduces costs to sell the wash composition as well as packaging waste.

The concentrated cleaning composition described exists as a liquid. The concentrated composition is formulated to be shelf stable, for example, not to undergo unexpected and/or determination changes during shipping, storage, etc. prior to use. In some embodiments, the concentrated composition is substantially free of solids. The concentrated composition may be substantially free of precipitates. The concentrated composition may remain free of precipitates and/or other solids during storage and/or environmental testing conditions to simulate storage.

“Free of”, as used herein in relation to a specific type of component, means that the referenced composition does not contain more than 0.5 wt. %, preferably no more than 0.1 wt. %, more preferably no more than 0.05 wt. % of said component relative to the total weight of the composition. Most preferably, said component is not contained at all.

As used herein, the term “substantially free” refers to compositions completely lacking the component or having such a small amount of the component that the component does not affect the performance of the composition. The component may be present as a minor constituent and/or impurity or contaminant and shall be less than 5 wt. %. In another embodiment, the amount of the component is less than 1 wt. % and in yet another embodiment, the amount of component is less than 0.1 wt. %.

Cleansing compositions typically contain an anionic surfactant for cleansing and foam generation, antibacterial agent, skin conditioning agents for cosmetic effects, dyes, perfumes, and optional thickening agents, such as clays, polymers, cellulosic derivatives, or colloids, for aesthetic effects, all in an aqueous carrier. However, the present compositions are drawn to compositions that are free of anionic surfactants and contain only cationic compatible surfactants.

The term “surfactant” or “surface active agent” refers to an organic chemical that when added to a liquid changes the properties of that liquid at a surface or interface.

As used herein, the term “cationic compatible” means a component that does not cause an insoluble complex with a cationic surfactant and/or does not substantially reduce the action of a cationic agent.

In various embodiments, the ready-to-use compositions contain water, typically in substantial amounts, such as up to 96 wt. %, preferably about 85 to about 95 wt. %.

When the cleaning composition is provided as a foam, the composition has a cellular structure that can be characterized as having several layers of air cells that provide the composition with a foamy appearance.

The composition according to the invention can be foamed without the use of a propellant. Mechanical foaming heads that can be used according to the invention to provide foam generation include those heads that cause air and the cleaning composition to mix and create a foamed composition. That is, the mechanical foaming head causes air and the cleaning composition to mix in a mixing chamber and then pass through an opening to create a foam.

It is generally expected that many compositions that contain a relatively large concentration of surfactant can be foamed when processed through a mechanical foaming head. When the concentration of surfactant is relatively low, such as less than 5%, it is often difficult to obtain sufficient foaming using a mechanical foaming head. It should be understood that sufficient foaming generally refers to the existence of a foam that provides a desired hang time or contact time when applied to a non-horizontal surface or that resists running or leveling for a desired length of time when placed on a horizontal surface. In the situation where the concentration of surfactant is relatively low, a foam-boosting or foam-stabilizing ingredients can assist in the generation of a desired foam when processed through a mechanical foaming head.

It has surprisingly been found that certain hydrophilic solubilizers will help generate sufficient foam in a ready-to-use cleaning composition containing less than 5% surfactant when prepared by diluting a concentrated composition containing the solubilizer, fragrance, benzoic acid or a salt thereof, cationic compatible surfactant. The solubilizer preferably has a HLB of 10-19 and is included in a concentrated composition in a ratio of about 1:1 to about 3:1, more preferably between 1.5:1 to about 2.7:1.

### Benzoic Acid

The compositions are preserved with benzoic acid or a salt thereof, preferably an inorganic salt. Benzoic acid and its salts effectively block yeast, bacteria, and mold with some exceptions.

The pKa of benzoic acid is 4.19-4.20. If the pH of the composition is lower, equilibrium is pushed closer to the non-dissociated state. The non-dissociated state is more effective against microorganisms.

Suitable salts of benzoic acid include alkali metal or alkali earth metal salts, such as potassium and sodium. Preferably, the salt is sodium.

The benzoic acid or salt thereof is included in a concentrated composition from about 0.5 to about 5 wt. %, more preferably about 1.5 to about 3.0 wt. %, most preferably about 1.8 to about 3.0 wt. % of the concentrated composition.

### Fragrance

The compositions described herein include a fragrance. Examples of possible fragrances include, but are not limited to natural oils or naturally derived materials, and synthetic fragrances such as hydrocarbons, alcohols, aldehydes, ketones, esters, lactones, ethers, nitriles, and polyfunctionals. Non-limiting examples of natural oils include the following: basil (Ocimum basilicum) oil, bay (Pimento acris) oil, bee balm (Monarda didyma) oil, bergamot (Citrus aurantium bergamia) oil, cardamom (Elettaria cardamomum) oil, cedarwood (Cedrus atlantica) oil, chamomile (Anthemis nobilis) oil, cinnamon (Cinnamomum cassia) oil, citronella (Cymbopogon nardus) oil, clary (Salvia sclarea) oil, clove (Eugenia caryophyllus) oil, cloveleaf (Eufenia caryophyllus) oil, Cyperus esculentus oil, cypress (Cupressus sempervirens) oil, Eucalyptus citriodora oil, geranium maculatum oil, ginger (Zingiber officinale) oil, grapefruit (Citrus grandis) oil, hazel (Corylus avellana) nut oil, jasmine (Jaisminum officinale) oil, Juniperus communis oil, Juniperus oxycedrus tar, Juniperus virginiana oil, kiwi (Actinidia chinensis) water, lavandin (Lavandula hybrida) oil, lavender (Lavandula angustifolia) oil, lavender (Lavandula angustifolia) water, lemon (Citrus medico limonum) oil, lemongrass (Cymbopogon schoenanthus) oil, lime (Citrus aurantifolia) oil, linden (Tilia cordata) oil, linden (Tilia cordata) water, mandarin orange (Citrus nobilis) oil, nutmeg (Myristica fragrans) oil, orange (Citrus aurantium dulcis) flower oil, orange (Citrus aurantium dulcis) oil, orange (Citrus aurantium dulcis) water, patchouli (Pogostemon cablin) oil, peppermint (Menthe piperita) oil, peppermint (Menthe peperita) water, rosemary (Rosmarinus officinalis) oil, rose oil, rose (Rosa damascene) extract, rose (Rosa multiflora) extract, rosewood (Aniba rosaeodora) extract, sage (Salvia officinalis) oil, sandalwood (Santalum album) oil, spearmint (Menthe viridis) oil, tea tree (Melaleuca altemifolia) oil, and ylang ylang (Cananga odorata) oil. Some non-limiting examples of synthetic hydrocarbon fragrances include caryophyllene, β-farnesene, limonene, α-pinene, and β-pinene. Some non-limiting examples of synthetic alcohol fragrances include bacdanol, citronellol, linalool, phenethyl alcohol, and α-terpineol (R=H). Some non-limiting examples of synthetic aldehyde fragrances include 2-methyl undecanal, citral, hexyl cinnamic aldehyde, isocycolcitral, lilial, and 10-undecenal. Some non-limiting examples of synthetic ketone fragrances include cashmeran, α-ionone, isocyclemone E, koavone, muscone, and tonalide. Some non-limiting examples of synthetic ester fragrances include benzyl acetate, 4-t-butylcyclohexyl acetate (cis and trans), cedryl acetate, cyclacet, isobornyl acetate, and α-terpinyl acetate (R=acetyl). Some non-limiting examples of synthetic lactone fragrances include coumarin, jasmine lactone, muskalactone, and peach aldehyde. Some non-limiting examples of synthetic ether fragrances include ambroxan, anther, and galaxolide. Some non-limiting examples of synthetic nitrile fragrances include cinnamonitrile and gernonitrile. Finally, some non-limiting examples of synthetic polyfunctional fragrances include amyl salicylate, isoeugenol, hedione, heliotropine, lyral, and vanillin.

The composition may include a mixture of fragrances including a mixture of natural and synthetic fragrances. The fragrance is present in a composition in an amount up to about 5 wt. %, preferably from 0.01 to about 3 wt. %, more preferably from about 1 to about 3 wt. %, and most preferably from about 1.5 to about 3.0 wt. %.

### Solubilizer

It was observed that the fragrance and benzoic acid or salt thereof can negatively interact with each other in concentrated compositions. However, inclusion of a hydrophilic solubilizer in the concentrate was found to minimize the interaction such that diluted compositions are able to produce high quality foams.

Suitable solubilizers are hydrophilic, preferably those having HLB 10-19, more preferably 10-17, most preferably about 10. Each surfactant and mixture of surfactants (and/or co-surfactants) has an HLB value that is a numerical representation of the relative weight percent of hydrophobic and hydrophilic portions of the surfactant molecule(s). HLB values are derived from a semi-empirical formula. The relative weight percentages of the hydrophobic and hydrophilic groups are indicative of surfactant properties, including the molecular structure, for example, the types of aggregates the surfactant will form and the solubility of the surfactant.

In general, surfactants with HLB values greater than 10 or greater than about 10 are called “hydrophilic surfactants.” Table 1 lists HLB values of exemplary solubilizers.

TABLE 1

| INCI Name | HLB |
|---|---|
| PEG-7 Glyceryl Cocoate | 10 |
| PEG-20 Almond Glycerides | 10 |
| PEG-25 Hydrogenated Castor Oil | 10.8 |
| Stearamide MEA | 11 |
| Glyceryl Stearate (and) PEG-100 | 11 |
| Polysorbate 85 | 11 |
| PEG-7 Olivate | 11 |
| Cetearyl Glucoside | 11 |
| PEG-8 Oleate | 11.6 |
| Polyglyceryl-3 Methyglucose Distearate | 12 |
| Oleth-10 | 12.4 |
| Oleth-10/Polyoxyl 10 Oleyl Ether NF | 12.4 |
| Ceteth-10 | 12.9 |
| PEG-8 Laurate | 13 |
| Cocamide MEA | 13.5 |
| Polysorbate 60 NF | 14.9 |
| Polysorbate 80 | 15 |
| Isosteareth-20 | 15 |
| PEG-60 Almond Glycerides | 15 |
| PEG-20 Methyl Glucose Sesquistearate | 15 |
| Ceteareth-20 | 15.2 |
| Oleth-20 | 15.3 |
| Steareth-20 | 15.3 |
| Steareth-21 | 15.5 |
| Ceteth-20 | 15.7 |
| Isoceteth-20 | 15.7 |
| Polysorbate 20 | 16.7 |
| Laureth-23 | 16.9 |
| PEG-100 Stearate | 18.8 |
| Steareth-100 | 18.8 |
| PEG-80 Sorbitan Laurate | 19.1 |

In certain embodiments, the solubilizer is a PEG ester or an ethoxylated sorbitan ester. PEG esters include, but are not limited to PEG-7 Glyceryl Cocoate, PEG-40 Hydrogenated castor oil, and PEG-60 Hydrogenated Castor Oil. Ethoxylated sorbitan esters include, but are not limited to, Polysorbate 20, Polysorbate 60, and Polysorbate 80.

In certain preferred embodiments, the solubilizer is PEG-7 Glyceryl Cocoate or Polysorbate 20. Most preferably, the solubilizer is PEG-7 Glyceryl Cocoate.

It is specifically noted that experiments utilizing polyols such as propylene glycol and glycerin failed to achieve the effect observed with hydrophilic solubilizers having HLB of about 10 or greater than 10.

The concentrated compositions of the invention include one or more hydrophilic solubilizing agents. These are present in the composition in an amount of from about 0.01 wt. % to about 5 wt. %, preferably from about 0.2 wt. % to about 1.5 wt. % and more preferably from about 0.75 wt. % to about 1.25 wt. %.

In certain preferred embodiments, the solubilizer is about 1.0 wt. % of a concentrated composition.

Preferably, the ratio of fragrance to solubilizer is about 1:1 to about 3:1, more preferably between 1.5:1 to about 2.7:1.

### Cationic Compatible Surfactant Component

The present foaming composition also contains one or more cationic compatible surfactants. Such surfactants include (a) nonionic surfactants (b) amphoteric surfactants, (c) cationic surfactants, and/or (d) zwitterionic, or mixtures thereof. The one or more cationic compatible surfactants is generally present in an amount of about 10 wt. % to about 40 wt. % preferably about 15 wt. % to about 30 wt. %, and more preferably from about 20 wt. % to about 25 wt. % of a concentrated composition.

Upon dilution, the surfactant component comprises about 1 to about 4 wt. % of a ready-to-use cleaning composition.

Nonionic Surfactants

Examples of nonionic surfactants include, but are not limited to, alcohol ethoxylates, fatty acid ethoxylates, alkyl phenol ethoxylate, monoalkonaolamide ethoxylates, sorbitan esters and their ethoxylated derivatives, ethoxylated fats and oils, amine ethoxylates, ethylene oxide-propylene oxide co-polymers, glycol esters, glycerol and polyglycerol esters, sucrose esters mono and polysaccharides surfactants, such as alkyl polyglucosides.

The antimicrobial composition can contain a nonionic surfactant component that includes a detersive amount of nonionic surfactant or a mixture of nonionic surfactants. Typically, a nonionic surfactant has a hydrophobic region, such as a long chain alkyl group or an alkylated aryl group, and a hydrophilic group comprising an ethoxy and/or other hydrophilic moieties.

In particular embodiments, the composition is free of an alkyl polyglucoside.

Amphoteric Surfactant

The cationic compatible surfactant component can include a detersive amount of amphoteric surfactant or a mixture of amphoteric surfactants. Amphoteric surfactants that can be used include, but are not limited to, imidiazolines and imidiazoline derivatives, betaine derivatives, amphoacetate derivatives, propionates, and mixtures thereof.

Exemplary betaine surfactants include those which may be represented by the general formula:

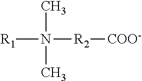

wherein R₁ is an alkyl group containing from 8 to 18 carbon atoms, or the amido radical which may be represented by the following general formula:

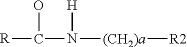

wherein R is an alkyl group having from 8 to 18 carbon atoms, a is an integer having a value of from 1 to 4 inclusive, and R2 is a C1-C4 alkylene group. Examples of such water-soluble betaine surfactants include dodecyl dimethyl betaine, as well as cocoamidopropylbetaine.

One or more amphoacetates such as sodium lauroamphoacetate, or diamphoacetates may also be used. Examples of such compounds include: sodium lauroamphoacetate, sodium cocoamphoacetate, disodium lauroamphoacetate, and disodium cocoamphoacetate.

In certain embodiments, the cleaning composition is free of an amphoacetate.

Preferably, a concentrated composition comprises at least one betaine present in at least 2% by weight of the composition. More preferably, a betaine comprises about 2.5 to about 5 wt. % of a concentrated composition.

In certain preferred embodiments, the at least one cationic compatible surfactant comprises cocoamidopropyl betaine (CAPB).

Cationic Surfactant

The surfactant component of the composition may also include a detersive amount of cationic surfactant or a mixture of cationic surfactants. Suitable cationic foaming agents contain quaternary ammonium groups. Cationic surfactants that can be used in the composition include, but are not limited to, quaternized sugar-derived surfactants, quaternized polysaccharides, quaternized alkyl polysaccharides, alkoxylated amines, alkoxylated ether amines, and mixtures thereof.

In certain embodiments, cationic surfactant/foaming ingredients include, for example, an organic salt of a quaternary ammonium containing compound or an inorganic salt of a quaternary ammonium containing compound or mixtures thereof.

Preferably, a concentrated composition comprises cationic surfactant present from about 2.5 to 15 wt. %, preferably about 5 to 10 wt. %, most preferably about 6 to 8 wt. %.

In certain embodiments, the at least one cationic compatible surfactant comprises cetrimonium chloride.

Zwitterionic Surfactant

The zwitterionic surfactants that can be used according to the invention include β-N-alkylaminopropionates, N-alkyl-β-iminodipropionates, imidazoline carboxylates, N-alkylbetaines, sulfobetaines, sultaines, amine oxides and polybetaine polysiloxanes.

Amine oxide may have a linear or mid-branched alkyl moiety. Typical linear amine oxides include water-soluble amine oxides containing one R1 C8-18 alkyl moiety and 2 R2 and R3 moieties selected from the group consisting of C1-3 alkyl groups and C1-3 hydroxyalkyl groups. The linear amine oxide surfactants in particular may include linear C10-C18 alkyl dimethyl amine oxides and linear C8-C12 alkoxy ethyl dihydroxy ethyl amine oxides.

Preferred amine oxides include linear C10, linear C10-C12, and linear C12-C14 alkyl dimethyl amine oxides. As used herein “mid-branched” means that the amine oxide has one alkyl moiety having n1 carbon atoms with one alkyl branch on the alkyl moiety having n2 carbon atoms. The alkyl branch is located on the a carbon from the nitrogen on the alkyl moiety. This type of branching for the amine oxide is also known in the art as an internal amine oxide. The total sum of n1 and n2 is from 10 to 24 carbon atoms, preferably from 12 to 20, and more preferably from to 16. The number of carbon atoms for the one alkyl moiety (n1) should be approximately the 5 same number of carbon atoms as the one alkyl branch (n2) such that the one alkyl moiety and the one alkyl branch are symmetric. As used herein “symmetric” means that (n1-n2) is less than or equal to 5, preferably 4, most preferably from 0 to 4 carbon atoms in at least 50 wt. %, more preferably at least 75 wt. % to 100 wt. % of the mid-branched amine oxides for use herein.

The amine oxide further comprises two moieties, independently selected from a C1-3 alkyl, a C1-3 hydroxyalkyl group, or a polyethylene oxide group containing an average of from about 1 to about 3 ethylene oxide groups. Preferably the two moieties are selected from a C1-3 alkyl, more preferably both are selected as a C1 alkyl.

In certain embodiments, the N-amine oxide is a C10-16 alkyldimethylamine oxide or lauramidopropyl amine oxide. Exemplary amine oxides are coco dimethyl amine oxide or coco amido propyl dimethyl amine oxide. In certain preferred embodiments, the n-amine oxide is lauramine oxide.

In some embodiments, an amine oxide is present in an amount of from about 0% to about 20% by weight of a concentrated composition. In some embodiments, the amine oxide is present in an amount of from about 5% to about 15%, or from about 10% to about 13% by weight of a concentrated composition.

Zwitterionic surfactants can also include betaines. Exemplary betaine surfactants include those which may be represented by the general formula:

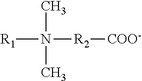

wherein R₁ is an alkyl group containing from 8 to 18 carbon atoms, or the amido radical which may be represented by the following general formula:

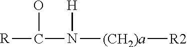

wherein R is an alkyl group having from 8 to 18 carbon atoms, a is an integer having a value of from 1 to 4 inclusive, and R2 is a C1-C4 alkylene group. Examples of such water-soluble betaine surfactants include dodecyl dimethyl betaine, as well as cocoamidopropylbetaine.

### Solvent

The carrier of compositions comprises water, propylene glycol, glycerols, alcohols or mixtures thereof. It should be appreciated that the water may be provided as deionized water or as softened water. The water provided as part of the composition can be relatively free of hardness. It is expected that the water can be deionized to remove a portion of the dissolved solids. That is, the concentrate can be formulated with water that includes dissolved solids, and can be formulated with water that can be characterized as hard water. The carrier present in the composition can be present in an amount of from about 30 wt. % to about 99 wt. %, preferably from about 55 wt. % to about 97 wt. % and more preferably from about 60 wt. % to about 75 wt. %.

A concentrated wash composition will contain less than 80 wt. % water, more preferably less than 70%, and most preferably less than 65% by weight water. Typically, a concentrated composition described herein will contain about 45 to about 60 wt. % water, preferably about 48 to about 60 wt. % water.

The concentrated compositions may contain additional non-aqueous solvent to improve the foaming of the composition. Not all solvents will necessarily function as foam-boosting solvents to cause a composition to foam when processed through a mechanical foaming head.

Exemplary non-aqueous solvents include glycols, glycol ethers, derivatives of glycol ethers, and mixtures thereof. Exemplary glycols include those having three carbon atoms such as glycerin and propylene glycol.

Exemplary glycol ethers include alkylene glycol ethers and aromatic glycol ethers. An exemplary aromatic glycol ether is ethylene glycol phenyl ether where R is a phenyl group, R′ is H, and n is a value of 1. Other exemplary glycol ethers include C₁-C₈ alkylene glycol ethers such as propylene glycol butyl ether, dipropylene glycol propyl ether, ethylene glycol butyl ether, diethylene glycol propyl ether, and triethylene glycol methyl ether. Exemplary derivatives of glycol ethers include those glycol ethers modified to include an additional group or functionality such as an ester group.

An exemplary range of non-aqueous solvent in a concentrated composition is between about 10 wt. % and about 20 wt. % of a concentrated composition.

In certain preferred embodiments, non-aqueous solvent will comprise or consist of glycerin and propylene glycol.

To accommodate inclusion of benzoic acid preservative, the concentrated compositions of the present invention have a pH of about 3.0 to about 5.5. Within this pH range, the present composition is consumer acceptable, i.e., provides adequate foam volume during wash generates stable, dense and rigid foam and is mild to skin and phase stable.

Preferably, pH of a concentrated composition ranges from 4.3 to 5.3, most preferably the pH of a concentrated composition is about 5.0.

In some instances, a pH adjusting compound may be necessary in a sufficient amount to provide a desired composition pH. To achieve the full advantage of the present invention, the pH-adjusting compound is present in an amount of about 0.05% to about 5.0%, by weight.

The identity of an acidic pH-adjusting compound is not limited and any acidic pH-adjusting compound known in the art, alone or in combination, can be used. Examples of specific acidic pH-adjusting compounds are the mineral acids and polycarboxylic acids. Nonlimiting examples of mineral acids are hydrochloric acid, nitric acid, phosphoric acid, and sulfuric acid. Nonlimiting examples of polycarboxylic acids are citric acid, glycolic acid, and lactic acid.

In certain embodiments, a concentrated composition contains citric acid as a pH adjusting agent.

In certain embodiments, malic acid is included in a concentrated composition as a pH adjusting agent.

### Other Ingredients

Chelating Agent

The storage properties of weak acids, such as benzoic acid, can be enhanced by adding chelating compounds to the compositions. The cleaning compositions herein can include a chelating agent. In general, a chelating agent is a molecule capable of coordinating (i.e., binding) the metal ions commonly found in water sources to prevent the metal ions from interfering with the action of the other ingredients. Examples of chelating agents include phosphonic acid and phosphonates, phosphates, aminocarboxylates and their derivatives, pyrophosphates, ethylenediamine and ethylenetriamine derivatives, hydroxyacids, and mono-, di-, and tri-carboxylates and their corresponding acids. In certain embodiments the composition is phosphate free.

Preferred chelating agents are selected from the group comprising ethylenediaminetetraacetic acid (EDTA); Ethylenediamine-N,N′-disuccinic acid (EDDS); diethylenetriaminepentacetic acid (DTPA); methylglycine-N,N-diacetic acid (MGDA); glutamic acid-N,N-diacetic acid (GLDA); Aspartic acid-N,N-diacetic acid (ASDA) and alkali, alkali earth metal, transition metal and/or ammonium salts thereof.

Dye

The composition may optionally include a dye. Examples of dyes include any water soluble or product soluble dye, any FD&C or D&C approved dye.

Antibacterial Agent

The concentrated compositions can optionally include one or more cationic antibacterial active ingredients.

Suitable cationic active ingredients contain quaternary ammonium group, such as alkyl dimethyl benzyl ammonium chloride (ADBAC, or benzalkonium chloride), alkyl dimethyl ethylbenzyl ammonium chloride, dialkyl dimethyl ammonium chloride, benzethonium chloride, N,N-bis-(3-aminopropyl) dodecylamine, chlorhexidine gluconate, a salt of chlorhexidene gluconate, PHMB (polyhexamethylene biguanide), salt of a biguanide, a substituted biguanide derivative, an organic salt of a quaternary ammonium containing compound or an inorganic salt of a quaternary ammonium containing compound or mixtures thereof.

For purposes of this disclosure, cetrimonium chloride is not considered as an antibacterial active agent.

Preferred cationic compatible antibacterial agents are chlorhexidine, benzalkonium chloride, and benzethonium chloride.

The amount of antimicrobial active agent is sufficient in the compositions of the invention to achieve a microbial kill in a short contact time, for example, 15 to 30 seconds.

When included, the antibacterial active agent will comprise from about 0.05 wt. % to about 5 wt. %, and preferably from about 0.4 wt. % to about 2 wt. %, and more preferably 0.5 wt. % to about 0.9 wt. % of a concentrated composition.

In certain preferred embodiments, the concentrated composition is substantially free of or free of an antibacterial active agent.

### Methods of Making the Compositions

It was discovered that forming a premix of fragrance and solubilizer then subsequently adding the premix to a mixture of the remaining ingredients of a concentrated composition, was able to best stabilize negative interactions between the fragrance and benzoic acid that can affect foam quality. The fragrance and solubilizer are preferably mixed until homogenous, generally for about 30 seconds to about 1.5 hours. The premix is mixed with remaining ingredients until homogeneous, generally at least 5 minutes to about 3 hours. Any suitable mixer used in the art may be used.

The compositions may be provided in various packaging sizes. Examples of packaging sizes include 25 mL, 0.84 oz, 221 mL, and 7.5 oz and can include bottles, ampules, pouches, sachets, vials and the like. Packages may include one or more smaller packages of a concentrated composition which may, or may not, be packaged along with a larger bottle used for diluting the concentrated composition and/or pumping a diluted composition. The selected packaging may have a pump head foamer. Examples of commercially available pump head foamers include various Palm Foamers from Rieke Corporation (Auburn, Indiana).

The concentrate can be diluted with water to provide the ready-to-use composition and/or the use composition. In general, it is expected that the concentrate will be diluted with water at a weight ratio of at least about 1:1. In addition, it is expected that the dilution of the concentrate with water will be less than about 1:20. Preferably, a concentrated composition is diluted with water in a ratio from about 1:4 to about 1:19, more preferably about 1:6 to about 1:9, most preferably 1:7 to 1:9. In addition, it is expected that the ready-to-use composition and/or the use composition will include at least about 90 wt. % water, preferably at least about 95 wt. % water, and more preferably at least about 96 wt. % water.

By providing the cleaning composition as a concentrate, it is expected that the concentrate will be diluted with the water available at the locale or site of dilution. It is recognized that the level of water hardness changes from one locale to another. Accordingly, it is expected that that concentrate will be diluted with water having varying amounts of hardness depending upon the locale or site of dilution. In general, water hardness refers to the presence of calcium, magnesium, iron, manganese, and other polyvalent metal cations that may be present in the water, and it is understood that the level of water hardness varies from municipality to municipality. The concentrated detergent composition is formulated to handle differing water hardness levels found in varying locations without having to soften the water or remove the hardness from the water. High solids containing water is considered to be water having a total dissolved solids (TDS) content in excess of 200 ppm. In certain localities, the service water contains a total dissolved solids content in excess of 400 ppm, and even in excess of 800 ppm. Water hardness can be characterized by the unit “grain” where one grain water hardness is equivalent to 17.1 ppm hardness expressed as CaCO₃. Hard water is characterized as having at least 1 grain hardness. Water is commonly available having at least 5 grains hardness, at least 10 grains hardness, and at least 20 grains hardness.

Advantageously, using a concentrated composition to refill a foam pump dispenser uses 95% less plastic than purchasing a ready-to-use formula in a new dispenser and can be ready to use in less than one minute. Concentrated refills can reduce carbon emissions in transportation by 85%.

### Methods Employing the Compositions

A method for foaming a cleaning composition described herein comprises providing a concentrated cleaning composition described herein, which minimally contains benzoic acid or a salt thereof, fragrance, at least one cationic compatible surfactant, hydrophilic solubilizer, and water. The concentrated cleaning composition is diluted to form a ready-to-use composition. The ready-to-use composition is diluted with air in a mechanical foaming head to generate a foam.

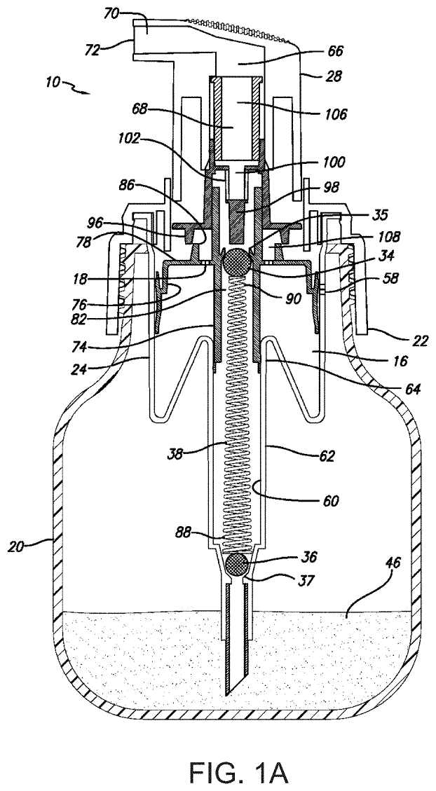

Now referring to FIGS. 1A and 1B, an exemplary foamer pump 10 for mixing a ready-to-use composition with air to form a foam and dispensing a ready-to-use cleaning composition has a fluid chamber 12, a mixing chamber 14, and an air chamber 16. The fluid chamber 12 contains a ready-to-use cleaning composition and has an inlet and an outlet. The outlet of the fluid chamber 12 is connected to the mixing chamber 14. The air chamber 16 has an air channel 108, which permits air to enter and exit the air chamber 16. The air channel 108 connects the air chamber 16 to the mixing chamber 14 in a second position of the foamer pump 10, and the air channel 108 connects the air chamber 16 to ambient air in a first position of the foamer pump 10. The air channel 108 has an air chamber passageway 18. The mixing chamber 14 provides a region for combining air from the air chamber 16 with the ready-to-use cleaning composition from the fluid chamber 12 to form an air/liquid mixture.

The foamer pump 10 further may have a fluid bottle 20 that contains the ready-to-use composition or a concentrated composition 46 that is ready for dilution, a closure 22, an accumulator 24, a liquid conduit 26, an actuator 28, a piston 30, a dip tube 32, an upper check valve 34, a lower check valve 36, a spring 38, a stem 40, an aerator 42, and an over-cap 44 The closure 22 is shaped and dimensioned to connect to the fluid bottle 20. The closure 22 has an upper edge 48. Preferably, the closure 22 has internal threads that mate with external threads on the neck of the fluid bottle 20.

The dip tube 32 is connected to the liquid conduit 26 and extends into the ready-to-use cleaning composition within the fluid bottle 20. The dip tube 32 provides a passage for transport of the ready-to-use composition from the fluid bottle 20 to the liquid conduit 26. The aerator 42 promotes foaming of the air/liquid mixture. The aerator 42 is located in the internal passage 66 of the actuator 28, between the mixing chamber 14 and the actuator outlet 72, and preferably within the axial portion 68 of the internal passage 66. The aerator 42 may contain one or more mesh screens 104 through which the air/liquid mixture is forced during the downward stroke to promote foaming of the air/liquid mixture.

In order to prepare a ready-to-use composition, a user can pour a concentrated cleaning composition 46 into an empty fluid bottle 20. The concentrated cleaning composition is then diluted with water by adding water to the bottle 20. The fluid bottle 20 may contain an indicator marking (not shown) to show the user how much water should be added to the concentrated composition. In certain preferred embodiments, an indicator marking indicates about 221 mL fill for the bottle 20. In a particularly preferred embodiment, about 25 g of a concentrated composition is dispensed into the bottle 20 and an additional 175 g to 215 g of water is added to the concentrate by the user. After water is added, the pump 10 is sealed onto the bottle via the closure 22 and the bottle is shaken or inverted at least several times, preferably 6 to 8 times, to prepare the ready-to-use composition 46.

The foamer pump 10 is activated by depressing the actuator 28 in the direction of the closure 22 in a downward stroke. Following the downward stroke, the foamer pump 10 is in second position, an example of which is shown in FIG. 1A. Removal of the depressing force (e.g., the user's finger pressure on the actuator 28) causes the actuator 28 to move in the direction away from the closure, 22 due to the force exerted by the spring 38 on the actuator 28, or an upward stroke. Following the upward stroke, the foamer pump 10 is back in a first position.

During the downstroke, the downward moving piston 30 causes the volume of the air chamber 16 to be reduced. As such, the air within the air chamber 16 is forced out of the air chamber passageway 18 and between the generally axial portion 92 of the stem 40 and the piston 30 so that the air may reach the mixing chamber 14.

During the downstroke, the downward moving piston 30 also reduces the volume of the fluid chamber 12. As such, liquid from the fluid chamber 12 is forced past the upper check valve 34 (which is unseated by the stem 40) and into the mixing chamber 14 for combination with the air from the air chamber 16.

Alternative embodiments of suitable foamer pumps and their use are described in U.S. Pat. No. 8,225,965, the contents of which are incorporated herein by reference.

### Embodiments

The following essential components of concentrated cleaning composition are exemplary of preferred embodiments within the scope of this disclosure that can stabilize negative interactions between benzoic acid and fragrance.

| | Minimum | Maximum |
|---|---|---|
| Ingredient | Active Wt % | Active Wt % |
| Water | 45 | 60 |
| Cationic Compatible Surfactant(s) | 10 | 40 |
| Benzoic Acid or Salt Thereof | 1.5 | 3.0 |
| Parfum (Fragrance) | 0.01 | 3.0 |
| Hydrophilic Solubilizer | 0.90 | 1.10 |

The concentrated composition should have a pH of between 3.0 and 5.5. Preferably, a ratio of the fragrance to the solubilizer is about 2:1 to about 3:1, more preferably about 2.3:1 to about 2.7:1, most preferably about 2.5:1.

### EXAMPLES

### Example 1: Concentrated Cleaning Compositions

Concentrated cleaning composition having ingredient makeup shown in Table 2 were prepared.

TABLE 2

| | Minimum | Maximum |
|---|---|---|
| Ingredient | Active Wt % | Active Wt % |
| Aqua (Water, Eau) | qs | qs |
| Glycerin | 10.8 | 13.1 |
| Cationic Compatible Surfactant | 20.2 | 25.0 |
| Organic Acid | 3.4 | 4.1 |
| Propylene Glycol | 2.7 | 3.3 |
| Sodium Benzoate | 1.8 | 3.0 |
| Fragrance | 0.01 | 2.9 |
| Solubilizer | 0.9 | 1.1 |
| Sodium Chloride | 0.6 | 0.7 |
| Tetrasodium EDTA | 0.2 | 0.2 |
| Bittering Agent | trace | trace |
| Dye | trace | trace |

The solubilizer was PEG-7 Glyceryl Cocoate, Polysorbate 20, Propylene Glycol, or Glycerin. The cationic compatible surfactant consisted of a mixture of lauramine oxide, cetrimonium chloride, and cocoamidopropyl betaine. Certain commercially available lauramine oxide products used contain trace amounts of dimethyl lauramine, dimethyl myristamine. The organic acid was citric acid.

A premix of the solubilizer and fragrance was prepared by mixing with Caframo laboratory stand mixer at 100 rpm with a four-blade propeller until homogenous for a minimum of 30 seconds to a maximum of 1.5 hours. The premix was added to the remaining ingredients and mixed until homogeneous for a minimum of 5 minutes to a maximum of 3 hours to prepare a concentrated cleaning composition.

195 g of water was added to 25 g of the compositions. The mixture was contained in a Reike F1 Dispensing Up-lock Foamer bottle and inverted 6-7 times. The foamer pump actuator was depressed to dispense foam.

Observations: PEG-7 Glyceryl Cocoate, Polysorbate 20, which are both hydrophilic solubilizer, produced suitable foams; the two hydrophilic solubilizers appeared to be interchangeable and no noticeable difference was observed in foam quality. However, the hydrophobic polyols did not produce foams.

The formulas having a ratio of fragrance to hydrophilic solubilizer of about 2.5:1 exhibited the best visual foam quality.

### Example 2: Effect of Fragrance and Preservative Level

Foams were prepared from concentrated compositions having a varied amount of fragrance or sodium benzoate preservative. The concentrated compositions are shown in TABLE 3. The cationic compatible surfactant consisted of a mixture of lauramine oxide, cetrimonium chloride, and cocoamidopropyl betaine. The organic acid was citric acid.

TABLE 3

| | |
|---|---|
| 2A | 2B |

| | |
|---|---|
| INCI Global | Wt. % |

| | | |
|---|---|---|
| Aqua (Water, Eau) | qs | qs |
| Glycerin | 11.9 | 11.9 |
| Cationic Compatible Surfactant | 22.4 | 22.4 |
| Organic Acid | 3.7 | 3.7 |
| Propylene Glycol | 3.0 | 3.0 |
| Sodium Benzoate | 2.95 | 2.0 or 2.95 |
| Fragrance | 0-2.36 | 2.36 |
| PEG-7 Glyceryl Cocoate | 1.0 | 1.0 |
| Sodium Chloride | 0.6 | 0.6 |
| Tetrasodium EDTA | 0.2 | 0.2 |
| Bittering Agent | trace | trace |
| Dye | trace | trace |

2A: About 25 g of the concentrated cleaning compositions containing 1.0% PEG-7 Glyceryl Cocoate solubilizer and various amounts of fragrance or were diluted with 175 to 215 g of water. The amount of fragrance was varied from 0% to 2.4%; the balance was adjusted with water. The diluted solutions were foamed using a Reike F1 Dispensing Up-lock Foamer and images of the resulting foam were captured using a LG G7 ThinQ phone camera.

2B: About 25 g of the concentrated cleaning compositions containing 1.0% PEG-7 Glyceryl Cocoate solubilizer and 2.0% or 2.95% sodium benzoate were diluted with 195 g of water; the balance was adjusted with water. The diluted solutions were foamed using a Reike F1 Dispensing Up-lock Foamer and images of the resulting foam were captured using a LG G7 ThinQ phone camera.

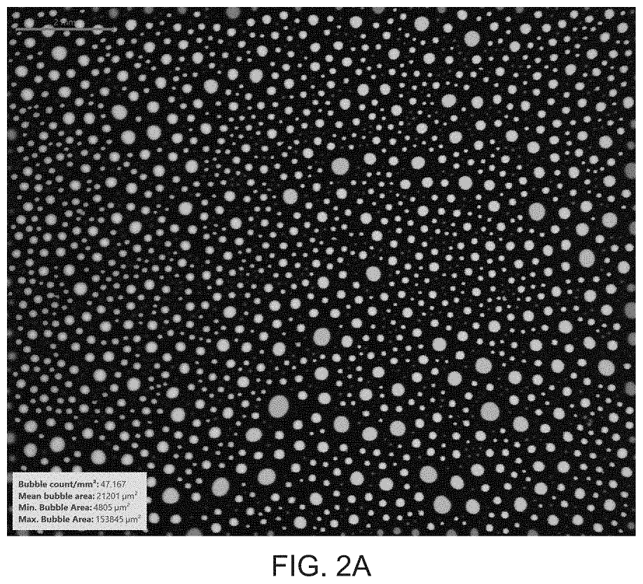

FIG. 3 shows images of the foams generated with various levels of fragrance at different dilution levels. Foams having increasing amounts of fragrance appeared to have larger micelles and a less creamy texture. It was observed that visual foam quality improves with decreasing fragrance weight percent and increased dilution.

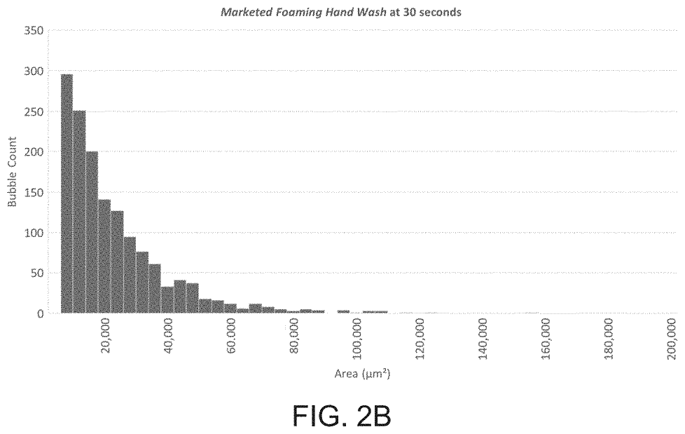

FIG. 4 shows images of foams generated from a composition containing 2.36% fragrance, 1% solubilizer and either 2.95 wt. % sodium benzoate (left) or 2.0 wt. % sodium benzoate (right). Larger micelles can be observed in the foam having 2.95% sodium benzoate. It was observed that foam quality improves with decreasing concentration of sodium benzoate.

### Example 3: Evaluation of Solubilizer Effect on Bubble Size and Count

Three compositions shown in TABLE 4 were further evaluated to understand effect of the solubilizer on the interaction between sodium benzoate and fragrance. The cationic compatible surfactant consisted of a mixture of lauramine oxide, cetrimonium chloride, and cocoamidopropyl betaine.

TABLE 4

| | | F100D100, | F100D100, |
|---|---|---|---|
| | F0D100 | solubilized | unsolubilized |
| INCI Global | Active Wt % | | |
| Aqua (Water, Eau) | 56.1 | 52.7 | 53.7 |
| Glycerin | 11.9 | 11.9 | 11.9 |
| Cationic Compatible Surfactant | 22.2 | 22.2 | 22.2 |
| Citric Acid | 3.7 | 3.74 | 3.74 |
| Propylene Glycol | 3.0 | 3.0 | 3.00 |
| Sodium Benzoate | 3.0 | 3.0 | 3.0 |
| Fragrance | 0 | 2.4 | 2.4 |
| PEG-7 Glyceryl Cocoate | 0 | 1.0 | 0 |
| Tetrasodium EDTA | 0.2 | 0.2 | 0.2 |
| Bittering Agent | trace | trace | trace |
| Dye | trace | trace | trace |

F0D100 having no fragrance was used as a control since it was determined that the undesirable interaction was coming from the combination of fragrance and sodium benzoate preservative. F100D100 solubilized contained a combination of fragrance and solubilizer while F100D100 unsolubilized did not contain the PEG-7 Glyceryl Cocoate solubilizer.

Viscosity of the concentrate was measured with Brookfield Ametek DV2T Viscometer LV-03 (63) Spindle, Speed: 60 rpm, Time: 1 minute.

Images of the foams were captured with a LG G7 ThinQ phone camera.

A Reike F1 Dispensing Up-lock Foamer was used to generate foams. The foams were generated from a ready-to-use formula containing 25 g of each of the above concentrated formulas diluted with 195 g water.

Particle size distribution of the foam was measured using Dynamic Light Scattering (DLS). A laser illuminates the sample while a fast photon detector analyses the scattered light from a known angle. This technique can be used to quantify particle size distributions for a variety of compositions including colloids, emulsions, and micelles. Dynamic Light Scattering (DLS) particle size measurement was obtained on a Brookfield NanoBrook Omni using the following settings:
Application: Particle Solutions v. 3.6.0.7122
Set duration: 120 seconds
Avg. Count Rate: >200.00 kcps
Angle: 90.0°
Cell type: square polystyrene cell
Wavelength: 640.0 nm
Particle size: 250 nm to 500 nm
Dust filter applied
Sample parameters: Liquid-water
Temp: 25° C.

A fluorescein indicator was used to show the relative number of micelles in each system and intensity was measured with a Molecular Devices SpectraMax i3x with the following parameters.
Application: Soft Max Pro 7 v 7.1.0 build 246936
Read Mode: Fluorescence
Read Type: Spectrum
Plate type: 96 well Corning half area clrbtm
Plate: Corning assay plate, 96 well, no lid, black, flat bottom,
polystyrene
Fuorescein (free acid) dye content 95%
Supplier: Aldrich
Dilution: 5×10⁻⁵ M
150 μl fluorescein, 150 μl sample in each well

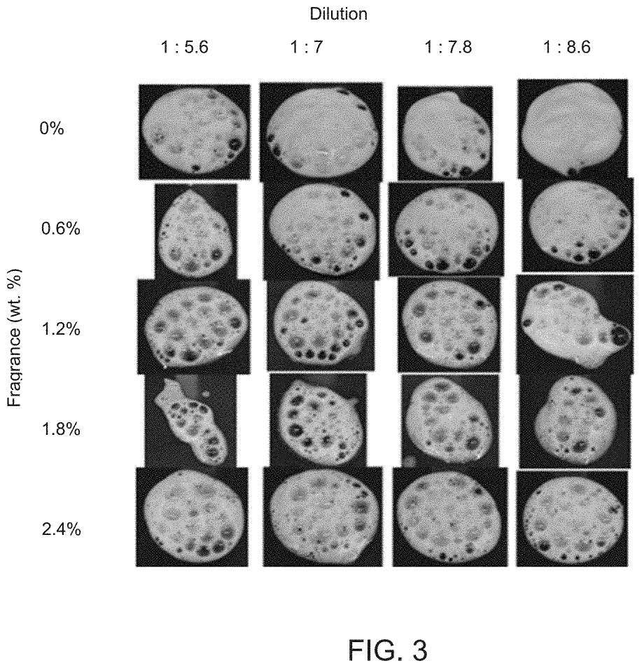

FIG. 5 shows particle size distribution obtained from DLS converted to a lognormal distribution. TABLE 5 provides the effective diameter of each foam and the viscosity of the concentrated composition.

TABLE 5

| | | Viscosity of |
|---|---|---|
| | Eff. Diameter of Foam | Concentrate |
| Sample ID | (nm) | (cPs) |
| F0 D100 | 19.16 | 40 |
| F100 D100, solubilized | 21.46 | 18 |
| F100 D100, unsolubilized | 22.72 | 50 |

For this format, where decreased viscosity is desired to pass through the foamer screen, a smaller micelle size is desirable. The results in FIG. 5 show that F100 D100, unsolubilized has the largest particle size. FO D100 with no fragrance has the smallest particle size. F100 D100, solubilized is between FO D100 and F100 D100, unsolubilized.

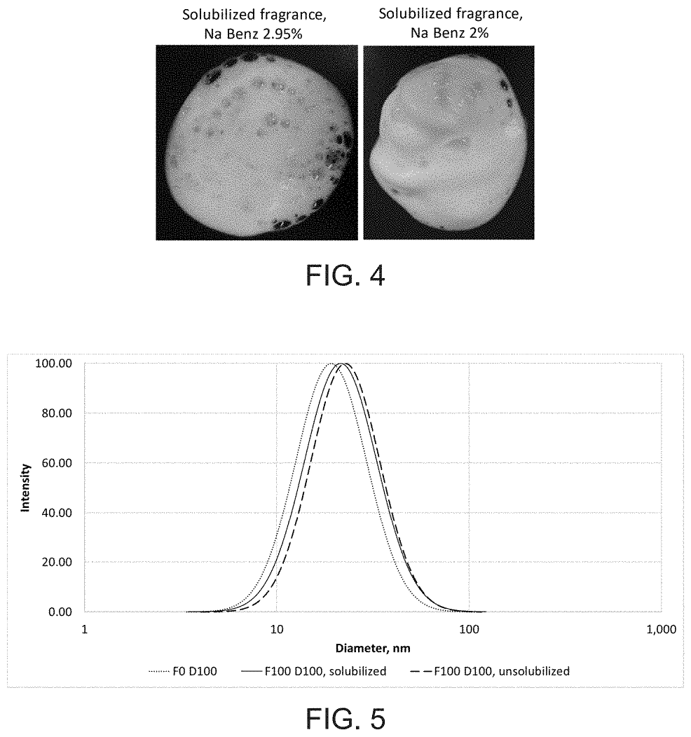

FIG. 6 shows the fluorescence intensity of the same three formulas shown in FIG. 5. The fluorescence measurements correlate with the particle size measurements. The sample with the most micelles was F0 D100 (no fragrance) and the one with the least was F100 D100, unsolubilized. The sample found in between was F100 D100, solubilized.

Based on the results found in FIGS. 5-6, one would expect FO D100 to have the best foam quality due to the small micelle size and the high number of micelles found in the formulation compared to the other two samples. Unexpectedly, when dispensing the three samples, F100 D100, solubilized had the best visual foam quality.

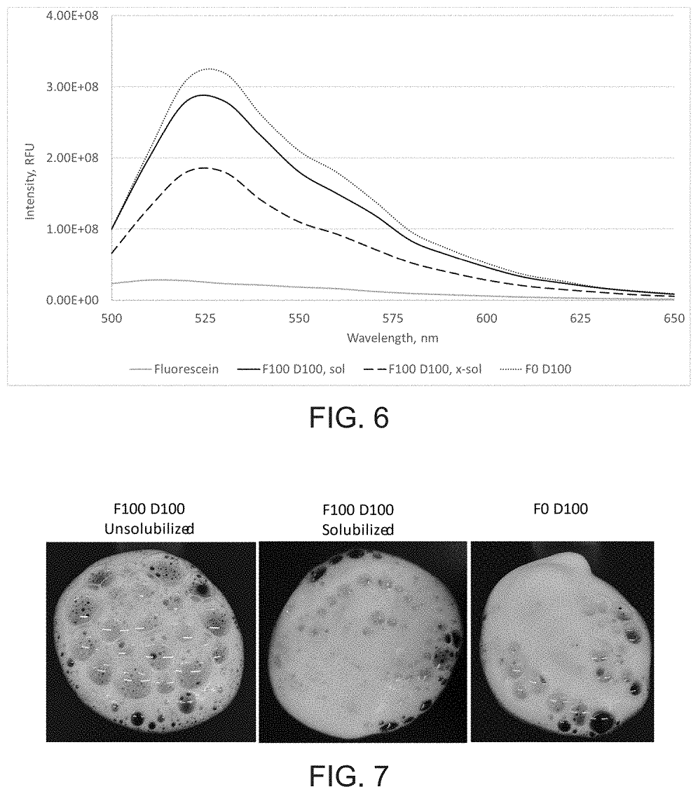

FIG. 7 shows visual foam quality comparison of the three formulas from FIGS. 5-6. F100 D100, solubilized has the smallest bubbles and is the most uniform when compared to the other samples.

### Example 4: Effect of Solubilizer on Foam Density, Mean Bubble Area, Bubble Size Distribution

To confirm and quantify the foam quality of the three concentrated formulas of Example 3 post-dilution, a Kruss Dynamic Foam Analyzer (DFA 100) was used to measure the foam density, mean bubble area, and bubble size distribution using the following parameters.
Column CY4501-40 mm
Height illumination: Blue-wavelength=469 nm
Camera position: 2
Camera height 70 mm
Frame rates:
Foaming Height: 5 fps
Foaming structure: 2 fps
Decay height: 2 fps
Decay structure: 2 fps
   Formula was dispensed directly into the column using the foaming hand wash pump, no sparging or agitation was used and the set duration was 120 s.

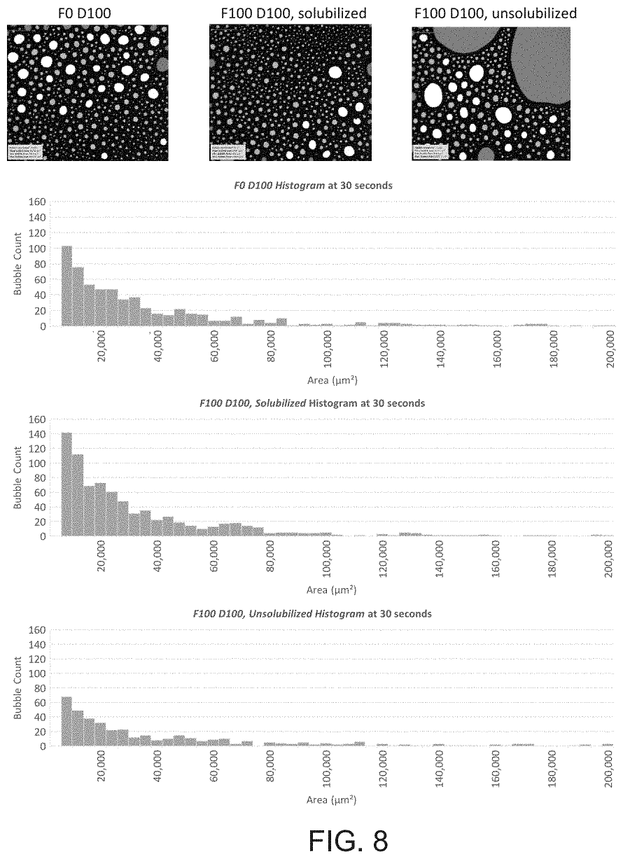

FIG. 8 shows images of the three foam samples measured using the foam analyzer along with their associated bubble size histogram. TABLE 6 contains the bubble count per millimeter and mean bubble area at 30 seconds values derived from the images in FIG. 8.

TABLE 6

| | Ideal | | F100 | F100 |
|---|---|---|---|---|
| | foaming | F0 | D100, | D100, |
| Sample ID | hand wash | D100 | solubilized | unsolubilized |
| Bubble count per mm² | >40 | 20.146 | 28.571 | 15.929 |
| Mean bubble area | <25000 | 49638 | 35001 | 62777 |
| (μm²) | | | | |

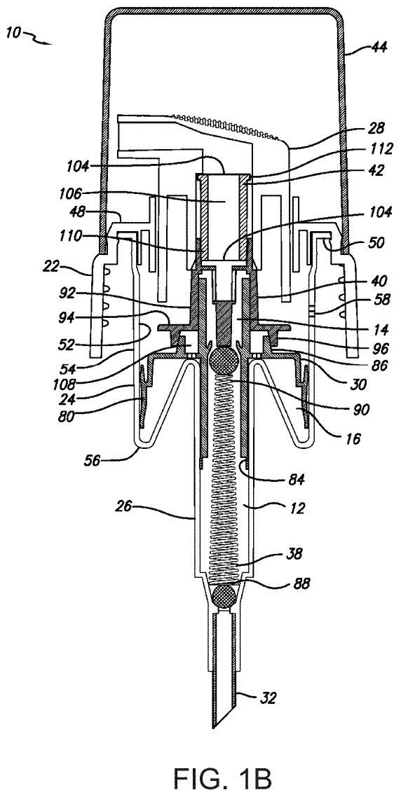

When comparing the three test samples, we found that F100 D100, solubilized has the highest bubble count per mm² and the smallest mean bubble area, and is the closest to the marketed foaming hand wash with high quality foam shown in FIG. 2 (bubble count was 47.167 mm⁻² and the mean bubble area was 21,201 μm² at 30 seconds). F100 D100, unsolubilized has the lowest bubble count per mm² and the largest mean bubble area and is furthest from the marketed foaming hand wash with high quality foam.

Inclusion of PEG-7 Glycerol Cocoate (HLB=10) solubilizer at 1% (ratio of fragrance to solubilizer of 2.4:1) was able to decrease the negative interaction between sodium benzoate and fragrance that causes lower foam quality. Unexpectedly, the formula with hydrophilic solubilizer produced better foam quality than a formula without fragrance. Similar results were observed with 1% Polysorbate 20.

It was concluded that inclusion of low levels of solubilizer having HLB 10-19 in a concentrate containing sodium benzoate and fragrance can be used to control and/or minimize negative interactions between the fragrance and benzoic acid when the fragrance is solubilized before addition to a concentrated cleaning composition preserved with benzoic acid. This improves the post-dilution performance of a foam prepared with the concentrate.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The breadth and scope of the present invention should not be limited by any of the above-described exemplary embodiments, but should be defined only in accordance with the following claims and their equivalents.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. A method for foaming a cleaning composition comprising:
providing a concentrated cleaning composition, the concentrated composition comprising:
benzoic acid or a salt thereof,
fragrance,
at least one cationic compatible surfactant,
a solubilizer having HLB 10-19, and
water present at about 45 to about 60 weight percent based on
a total weight of the concentrated composition;
diluting the concentrated cleaning composition to form a ready-to-use composition; and
mixing the ready-to-use composition with air in a mechanical foaming head to generate a foam.

2. The method of claim 1, wherein a ratio of the fragrance to the solubilizer is about 1:1 to about 3:1.

3. The method of claim 3, wherein the ratio of the fragrance to the solubilizer is about 1.5:1 to about 2.7:1.

4. The method of claim 1, wherein the diluting occurs in a single dilution step.

5. The method of claim 1, wherein the diluting occurs in multiple dilution steps.

6. The method of claim 1, wherein diluting comprises diluting with water at a dilution ratio of about 1:4 to about 1:9 parts concentrated cleaning composition to water.

7. A stable concentrated foaming cleaning composition comprising:
benzoic acid or a salt thereof;
fragrance;
at least one cationic compatible surfactant;
solubilizer having HLB 10-19; and
water present at about 45 to about 60 weight percent based on a total weight of the concentrated composition, wherein a ratio of the fragrance to the solubilizer is about 1:1 to about 3:1.

8. The concentrated composition of claim 7, wherein the at least one cationic compatible surfactant comprises about 10 to about 40 percent by weight of the composition.

9. The concentrated composition of claim 7, wherein the benzoic acid or a salt thereof comprises about 1.5 to about 3.0 percent by weight of the concentrated composition

10. The concentrated composition of claim 7, wherein the fragrance comprises about 0.01 to about 3.0 percent by weight of the concentrated composition.

11. The concentrated composition of claim 7, wherein the solubilizer comprises about 0.2 to about 1.5 percent by weight of the concentrated composition.

12. The concentrated composition of claim 7, wherein the solubilizer is PEG-7 Glyceryl Cocoate or Polysorbate 20.

13. The concentrated composition of claim 7, further comprising an antibacterial active agent.

14. A method of improving quality of a foam produced from a concentrated foaming cleaning composition containing fragrance, benzoic acid or a salt thereof and at least one cationic compatible surfactant comprising:
mixing the fragrance with a solubilizer having HLB 10-19 to form a fragrance premix; and
adding the fragrance premix to other ingredients of the concentrated foaming cleaning composition.

15. The method of claim 14, wherein the solubilizer is PEG-7 Glycerol Cocoate or Polysorbate 20.

16. The method of claim 14, wherein the fragrance comprises about 0.01 to about 3.0 percent by weight of concentrated foaming cleaning composition.

17. The method of claim 14, wherein the solubilizer comprises about to 0.2 about 1.5 percent by weight of the concentrated foaming cleaning composition.

18. The method of claim 14, wherein the benzoic acid or a salt thereof comprises about 0.5 to about 5 percent by weight of the concentrated foaming cleaning composition.

19. The method of claim 14, wherein a ratio of the fragrance to the solubilizer is about 1:1 to about 3:1.

20. The method of claim 14, wherein dilution of the concentrated composition with water in a 1:4 to 1:9 ratio of concentrated composition to water followed by mixing with air in a mechanical foaming head produces a smaller mean bubble area and a greater bubble count per mm² than a foam prepared from a concentrated cleaning composition having no solubilizer.
